SEQUENCE LISTING (2 sequences)

SEQ ID NO: 1 - protein, 425 aa, Homo sapiens
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

SEQ ID NO: 2 - protein, 8 aa, Homo sapiens
DYKDDDDK

(19) United States
(12) Patent Application Publication
(10) Pub. No.: US 2006/0276386 A1
(43) Pub. Date: Dec. 7, 2006

(54) Use of substances inhibiting the association of said protein with ubiquitin c-terminal hydrolase for altering cellular responses to tgf-beta or bmp

(76) Inventors: Andrew Chantry, Norwich (GB); John Stephen Wicks, Norwich (GB)

Correspondence Address: MORGAN LEWIS & BOCKIUS LLP, 1111 PENNSYLVANIA AVENUE NW, WASHINGTON, DC, 20004, US

(21) Appl. No.: 10/542,405

(22) PCT Filed: Jan. 30, 2004
(86) PCT No.: PCT/GB04/00395

§ 371 Date: May 8, 2006

(30) Foreign Application Priority Data
Feb. 1, 2003 (GB) 0302315.7

(51) Int. Cl. A61K 38/17

(52) U.S. Cl. 514/12

(57) ABSTRACT

Disclosed is a pharmaceutical composition for altering cellular responses to TGFβs and/or BMPs; the composition comprising a molecule which prevents, inhibits or reduces the association of a Smad protein with a UCH, in admixture with a physiologically acceptable carrier, excipient or diluent.

### FIELD OF THE INVENTION

The present invention relates to methods of regulating responses, in vivo or in vitro, to hormones of the TGFβ superfamily, to pharmaceutical compositions for such purposes, a method of making a pharmaceutical composition, and to use of certain substances to regulate responses to hormones of the TGFβ superfamily.

### BACKGROUND OF THE INVENTION

There is a group of secreted polypeptide hormones known collectively as the Transforming Growth Factor β (TGFβ) superfamily.

TGFβs control a broad range of normal biological activities including cell growth, bone development, cell migration, differentiation and apoptosis. However, aberrant TGFβ signalling is responsible for a number of developmental disorders, human cancers and other diseases (see, for example, Massague et al., 2000 Cell 103, 295-309).

Recently the signal transduction pathways, by which cells detect and respond to the presence of TGFβs have been at least partially elucidated, including the intracellular components which transduce TGFβ signals into the cell nucleus (reviewed by Moustakas et al, 2001 J. Cell Sci. 114, 4359-4369).

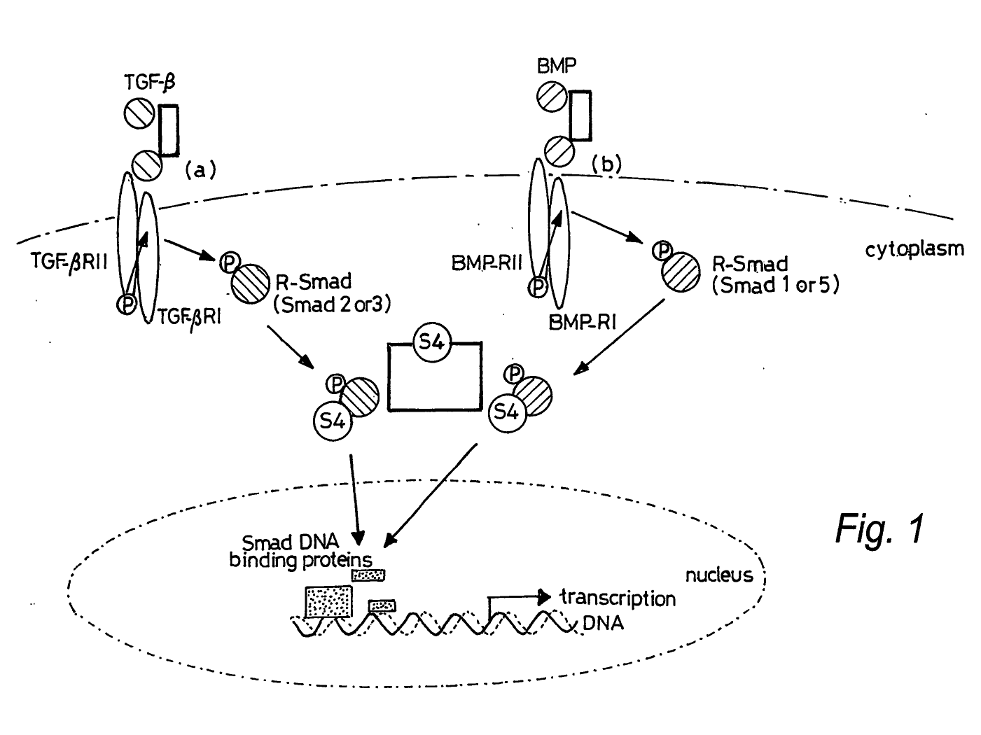

Genetic screens in Drosophila isolated a protein called MAD (‘mothers-against-decapentaplegic’) due to its involvement in the TGFβ signalling pathway known as Decapentaplegic (dpp). MAD-related proteins were subsequently identified in vertebrates and designated as Smad proteins. Smad proteins act downstream of the transmembrane serine-threonine kinase receptors that mediate TGFβ signals (see FIG. 1). To date, 10 members of the Smad family have been described, and can be segregated into three functionally distinct sub-groups.

Upon activation, the TGFβ receptor complex induces phosphorylation of the receptor-regulated R-Smads (Smads 1, 2, 3, 5, 8). Receptors for TGFβ can activate Smad2, Smad3 and Smad8, and receptors for related factors (Bone morphogenic proteins, BMPs) activate Smad1 and Smad5. In the unstimulated state, R-Smads are maintained in an inactive conformation by internal interactions between conserved N-terminal Mad homology 1 (MH1) and C-terminal Mad homology 2 (MH2) domains. Phosphorylation of the C-terminal -Ser-Ser-X-Ser- motif in receptor-regulated Smads disrupts these auto-regulatory MH1-MH2 domain intramolecular interactions to facilitate Smad activation. In all cases, the phosphorylated R-Smads then associate with a common-mediator or co-Smad (Smad4). These heteromeric complexes are translocated to the nucleus, where they regulate gene transcription by either association with DNA-binding proteins or direct binding to promoter sequences in target genes.

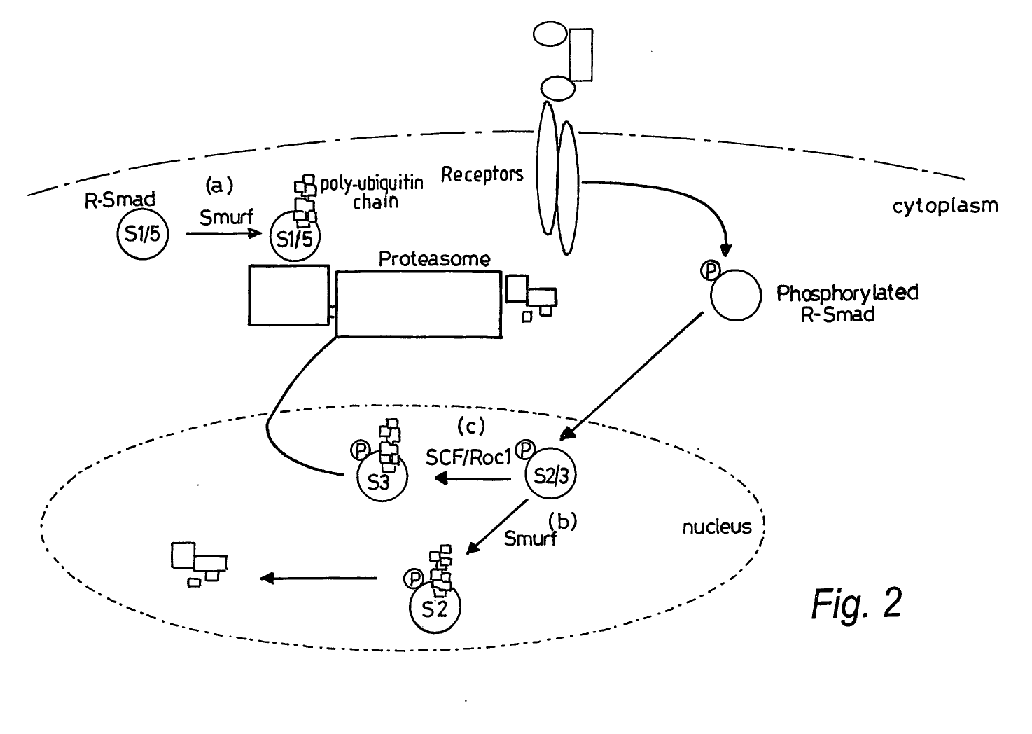

Regulation of TGFβ signalling is effected, in part, by a feedback mechanism that involves specific protein ubiquitination and proteasomal degradation of Smads. Ubiquitination plays a key role in a number of biological processes including signal transduction, cell cycle, and gene expression (Wilkinson, 2000 Cell Develop. Biol. 11, 141-148). Ubiquitination of proteins involves the concerted action of an E1 ubiquitin-activating enzyme, E2 ubiquitin conjugating enzymes, and E3 ubiquitin ligases that play a role in the specific recognition of target substrates. Recently, a new type of E3-type ubiquitin ligases, known as Smurfs, have been shown to bind to Smads and have been implicated in their specific ubiquitination (see FIG. 2). Smurf1 can interact selectively with Smad1 (BMP pathway specific), and this mechanism appears to regulate the abundance of Smad1 in unstimulated cells since it is not affected by receptor activation. Smurf2 has been shown to interact with Smads 1, 2 and 3, however, only Smad2 becomes ubiquitinated and degraded by proteasomes. In this instance, Smad2 interaction with Smurf2 is dependent upon receptor activation and the C-terminal phosphorylation of Smad2. In all cases, a small region in Smurfs known as a WW domain is responsible for the interaction with a -Pro-Pro-X-Tyr- sequence motif in Smads. Smad3 also undergoes ubiquitination by the SCF/Roc1 E3 ligase complex and subsequent degradation in the proteasome (Fukuchi et al, 2001 Mol. Biol. Cell 12, 1431-1443). In this instance, the ubiquitin ligase binds to a region in the C-terminal MH2 domain that is distant from the -Pro-Pro-X-Tyr- sequence motif in Smad3.

In view of the role that inappropriate TGFβ-induced responses play in a large number of diseases it would be useful to have an alternative means of regulating TGFβ signalling. Such an alternative is provided by the present invention.

The content of all publications mentioned in this specification is specifically incorporated herein by reference.

### SUMMARY OF THE INVENTION

The present inventors have identified a previously unknown interaction between Smad proteins and ubiquitin C-terminal hydrolases (UCHs). UCHs are enzymes which, as their name suggests, cleave ubiquitin. At least some UCHs are already well-characterised (see, for instance, Johnston et al, 1997 EMBO J. 16, 3787-3796; and Johnston et al, 1999 EMBO J. 18, 3877-3887). The association of Smad proteins with UCHs is thought likely by the inventors to result in stabilisation of the Smad, by inhibiting ubiquitin-mediated proteasomal degradation. Thus any method of preventing, inhibiting or reducing the association between Smads and UCHs should result in alteration of cellular responses to TGFβs and/or Bone morphogenic proteins (BMPs).

Accordingly, in a first aspect the invention provides a method of altering (especially down-regulating) cellular responses to TGFβs and/or BMPs, the method comprising the step of introducing into a cell a molecule which prevents, inhibits or reduces the association of Smad proteins with UCHs. The method may be performed on cells in vitro or in vivo.

In a second aspect the invention provides for use of a molecule which prevents, inhibits or reduces the association of a Smad protein with a UCH, for the alteration (preferably down-regulation) of cellular responses to TGFβs and/or BMPs.

In a third aspect the invention provides for use of a molecule which prevents, inhibits or reduces the association of a Smad protein with a UCH in the preparation of a medicament to alter (preferably down-regulate) cellular responses to TGFβs and/or BMPs.

In a fourth aspect the invention provides a pharmaceutical composition for altering (preferaby down-regulating) cellular responses to TGFβs and/or BMPs, the composition comprising a molecule which prevents, inhibits or reduces the association of a Smad protein with a UCH, in admixture with a physiologically acceptable carrier, excipient or diluent.

In a fifth aspect the invention provides a method of screening a test substance for the ability to prevent, inhibit or reduce the association of a Smad protein with a UCH, the method comprising the step of contacting the test substance with a Smad protein and/or a UCH and determining, qualitatively or quantitatively, the amount of association of the Smad protein with the UCH when these are contacted. The determination may be made in absolute or relative terms. Conveniently one or more of the test substance, Smad protein and UCH may be labelled with a readily detectable label such as a radio label, fluorophore, chromophore, enzyme, antibody or the like. In a particular embodiment the method of screening may make use of, for example, a cell or cell extract. Test substances identified by the screening method may be of potential usefulness as drugs to alter (preferably down-regulate) cellular responses to TGFβs and/or BMPs. The screening method of the invention may conveniently comprise one, two or all of the following: ELISA; co-immunoprecipitation; Western blotting.

In one particular embodiment the invention applies specifically to the (preferably down-regulation) of responses to TGFβs rather than to BMPs, by preventing, inhibiting or reducing the association of UCHs with Smad3, which protein is involved in transduction of TGFβ signals but not transduction of BMP signals. Thus the invention especially relates, in particular embodiments, to methods, uses and compositions for preventing, inhibiting or reducing the association between Smad3 and UCHs.

In particular, the association of Smad3 is believed to be strongest with UCH-L5 (a mouse UCH), or with the corresponding human homologue UCH37. Thus the invention in particular embodiments relates to a method of or composition for preventing, inhibiting or reducing the association between Smad3 and UCH-L5 or UCH37.

The present invention contemplates the use of any molecule which can have the desired effect, which may particularly be achieved, for instance, by:
(i) using a molecule which comprises a structural analogue of the UCH-binding site on Smad proteins which can therefore inhibit (reversibly or irreversibly) or interfere with UCH binding to Smads;
(ii) using a molecule which comprises a structural analogue of the Smad-binding site on UCHs (localised to at least the N terminal 195 amino acid residues of, for example, UCH-L5), which can therefore inhibit (reversibly or irreversibly) or interfere with Smad proteins binding to UCHs;
(iii) using a molecule which (preferably specifically) reduces the effective intracellular concentration of SMAD or (more preferaby) UCH e.g. by promoting degradation of UCHs. An example of such a molecule is ubiquitin aldehyde (or Uba1). Ubiquitin aldehyde is a ubiquitin derivative in which the C terminal carboxylate group is replaced by an aldehyde, and is a potent inhibitor of UCHs. (See, for example, Johnston et al, 1999 EMBO J. 18, 3877-3887; and Hu et al, 2002 Cell 111, 1041-1054.)

Molecules which may be useful in preventing or inhibiting the interaction of a Smad protein with a UCH, and in particular a molecule within category (i) or (ii) above, may be prepared by a rational drug design approach. To this end, the inventors propose to form a crystal of a complex between a Smad and a UCH (in particular Smad3 and a C-terminally truncated UCH such as UCH-37. (The C terminal truncation results in a stronger intraction between the proteins and facilitates crystallization).

Crystallization techniques are now a matter of routine for those skilled in the art. For example, standard techniques are taught by McPherson (Eur. J. Biochem. 189, 1-23; and “Crystallization of Biological Macromolecules” (Trends in Cell Biology) 1999 Cold Spring Harbor Laboratory Press) and by Ducruix & Griege (Eds.) “Crystallization of Nucleic Acids and Proteins: A Practical Approach” (1996 Irl Press).

In addition Smad polypeptides have already been crystallised (Shi et al, 1998 Cell 94, 585-594; Wu et al, 2001 Molec. Cell 8, 1277-1289) as have UCH molecules (Johnston et al, 1997 EMBO J. 16, 3787-3796; Johnston et al, 1999 EMBO J. 18, 3877-3887; & Hu et al; 2002 Cell 111, 1041-1054) and this information should facilitate the crystallization of a Smad/UCH complex as proposed by the inventors. Once the complex has been crystallised it can be subjected to structural analysis at the atomic level by X-ray crystallography. Such techniques are now routine for those skilled in the art. The resulting data provide detailed information on the structure of the complex, which can be input into various commercially available computer programs to derive, in a rational manner, structures of small molecules (e.g. peptides) which should be able to block or inhibit the interaction of Smads with a UCH.

The interaction between Smad and UCH molecules may also be studied using molecular modelling methods. Comparative modelling may be used to generate models of the Smad and UCH polypeptides being studied, based on their homology with Smad and UCH molecules of known structure. An example of a modelling program that may be used is MODELLER (Sali et al, 1995 Proteins 23, 318-326). The lowest energy models generated using MODELLER may then be further refined using techniques such as energy minimisation and molecular dynamics. Following refinement, the data obtained from the lowest energy models may then be used to assess the interaction between Smad and UCH using a docking program, such as 3D-DOCK (Smith & Sternberg, 2003 Proteins 52, 74-79), to generate a model of the Smad/UCH complex.

The resulting data from the three-dimensional crystal structure and/or model provide detailed information on the structure of the complex. In particular, the structural information allows the determination of the residues involved in the interaction which may then be used to design small molecules (e.g. peptides) which should be able to block or inhibit the interaction of Smads with a UCH.

Molecules designed in this way can then be synthesised and tested in vitro for relevant activity in preventing or inhibiting Smad/UCH interaction, using an in vitro assay along the lines disclosed in the present specification.

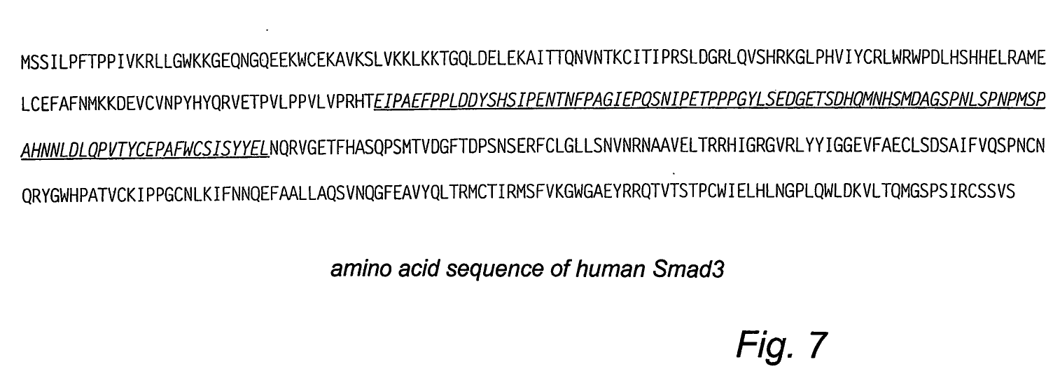

More specifically within molecules of category (i), the inventors have been able to establish that a portion of Smad3 present within residues 144-240 are essential for UCH-L5 or UCH37 to bind to Smad3. The sequence of human Smad3 has been published (Nature vol. 383, 1996 p 168-172) and is available from Genbank (accession no. U68019). The amino acid sequence of the human Smad3 protein is shown, using single letter code, in FIG. 7 (Seq. ID No. 1). Residues 144-240 are shown italicised and underlined.

Thus, in some embodiments, the method of the invention may comprise introduction into the cell (within which the response to TGFβ is to be altered) of a molecule which comprises a peptide having at least 60% sequence identity, preferably at least 70%, more preferably at least 80%, and most preferably at least 90% sequence identity with a contiguous portion of Smad3, which portion is preferably present within amino acid residues 144-240 of Smad3. Typically the molecule introduced into the cell will comprise a peptide of at least 8 amino acid residues having the desired level of sequence identity with the corresponding contiguous portion of Smad3, preferably at least 10 amino acid residues, more preferably at least 12 amino acid residues and most preferably 15 or more amino acid residues. The peptide will generally not comprise the full length Smad protein, and certainly not a signalling-competent Smad moiety, otherwise the object of the invention will be defeated. The peptide will preferably comprise no more than 80 amino acid residues, more preferably no more than 60 amino acid residues, and most preferably no more than 40 amino acid residues.

The molecule may comprise modified or non-naturally occurring amino acid residues and/or non-peptide moieties in order to optimise the pharmacokinetic characteristics (e.g. increase stability [e.g. resistance to protease-mediated degradation]; reduce toxicity, and/or increase bioavailability). For example, the molecule may comprise a lipid or other hydrophobic moiety in order to improve transport across the cell membrane. Alternatively the molecule could be incorporated into or within a particulate vector, such as a liposome. Numerous suitable liposomes are known to those skilled in the art.

Where the molecule of use in the method consists of a peptide or small protein, it may be preferable to introduce into the cell a nucleotide sequence (typically a DNA sequence) which directs the expression in the cell of the effector peptide or protein. Nucleotide sequences can be introduced into cells in vitro or in vivo by a number of well known techniques including transfection, transduction by viral vectors (e.g. vaccinia virus and modified vaccinia virus ankara [MVA], adenovirus and the like), and by use of “gene guns” and so on.

Molecules which specifically reduce the effective intracellular concentration of UCHs may include UCH-specific proteases or molecules which interfere with the expression of UCHs. In this respect UCH-specific ribozymes or RNAi approaches may usefully be employed.

RNA interference (RNAi) is the name given to the phenomenon whereby the presence in a cell of double-stranded RNA can lead to sequence-specific degradation of mRNA, leading to inhibition of expression of a specific gene or genes.

Detailed guidance on the design of appropriate oligonucleotides for use in RNAi is available, inter alia, on the Qiagen website (www.qiagen.com). For example, it is known that dsRNA oligonucleotides of 21-23 bases in length work well (Elbashir et al, 2001 Nature 411, 494); the selected target mRNA sequence (complementary to the introduced ds RNA) should have a GC ratio as close to 50% as possible; the target mRNA should preferably be selected to avoid comprising four or more contiguous guanosines or contiguous cytosines. In addition, Qiagen offer custom synthesis of RNAi Oligonucleotides.

The pharmaceutical composition of the invention may be administered to a human or animal (preferably mammalian) subject by any convenient means: orally; by injection—intravenously, subcutaneously or, intramuscularly; intranasally; topically; rectally and so on. The composition may take the form of an injectable solution, a suspension, a spray, a cream, ointment, gel, dry powder, tablet, pill, capsule or the like.

Typically the pharmaceutical composition may comprise the active agent at a concentration in the range 0.01 mg/gm to 100 mgs/gm, more preferably in the range 0.1 mg/gm to 10 mgs/gm. A suitable dose can readily be ascertained for a particular subject by trial-and-error—a minimal dose may be administered for say 24-48 hrs, and the dose gradually increased (typically in a stepwise manner) until a therapeutic benefit or an adverse reaction is observed. A therapeutic benefit may be defined as any improvement in a subject's clinical condition which is recognisable by a suitably-qualified health professional and/or may readily be quantified relative to any absolute or relative index (e.g. size of a tumour).

For the avoidance of doubt it is hereby expressly stated that any feature described in this specification as “preferred”, “desirable”, “advantageous”, “convenient” or the like may be adopted in any embodiment of the invention in isolation or in combination with any other feature of the invention so-described, unless the context dictates otherwise. Further, unless the context dictates otherwise, features which are preferable in relation to one aspect of the invention will generally be preferable in relation to other aspects of the invention.

The invention will now be further described by way of illustrative example and by reference to the accompanying drawings, in which:

FIG. 1 is a schematic representation of the TGFβ and BMP signalling transduction pathways in a eukaryotic cell:

FIG. 2 is a schematic representation of the ubiquitin-mediated proteasomal degradation of Smad proteins;

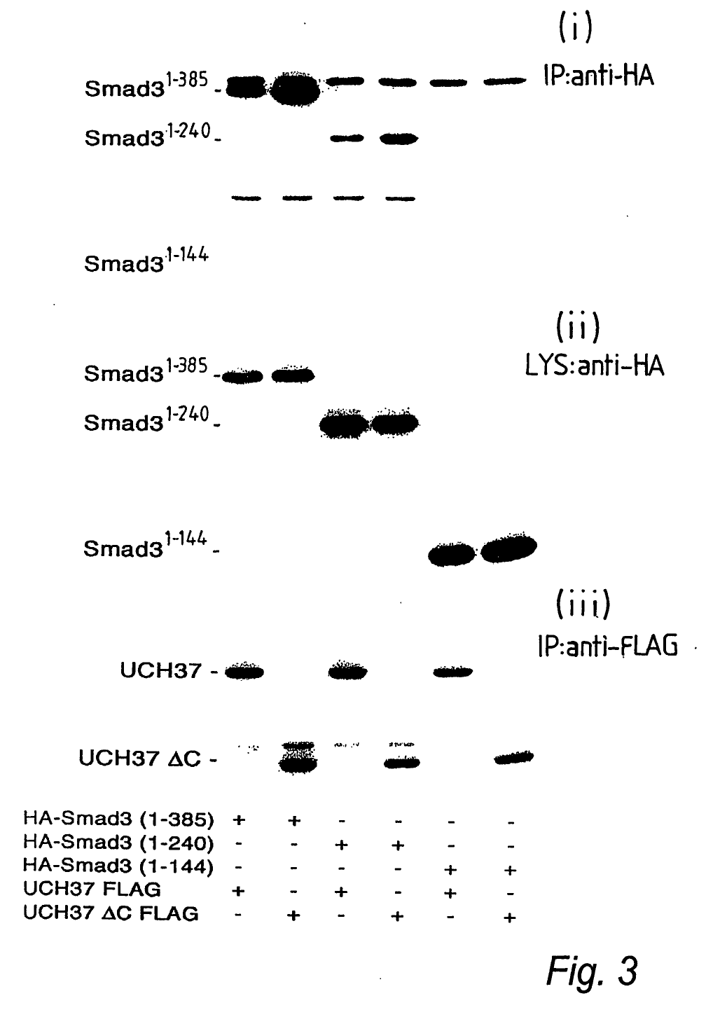

FIGS. 3(i)-(iii), 8 and 9 are pictures showing the results of various immuno-precipitation experiments;

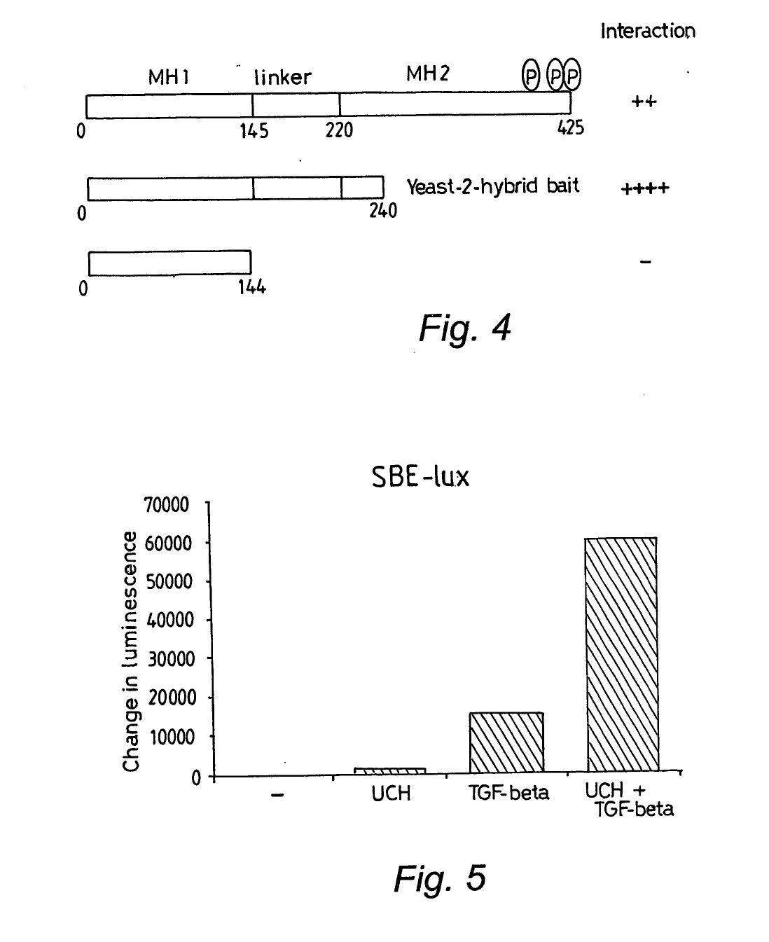

FIG. 4 is a schematic representation of Smad3 protein and various truncations thereof employed by the inventors, together with an indication of their relative strength of interaction with UCH37;

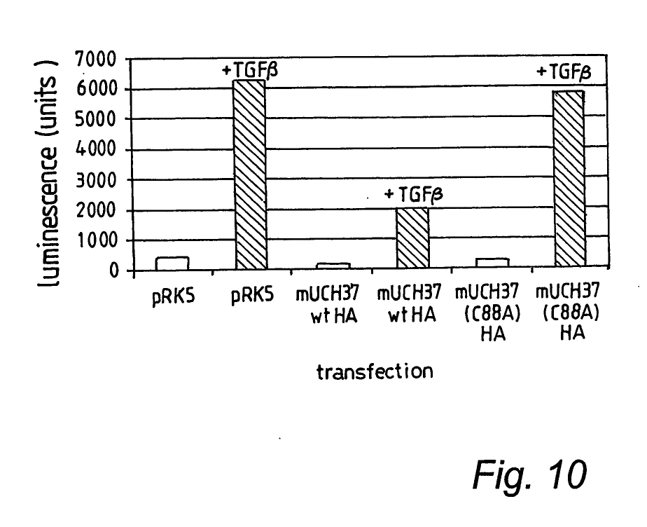

FIGS. 5 and 10 are bar charts of change in luminescence (arbitary units) for cells transfected with different combinations of nucleic acid constructs;

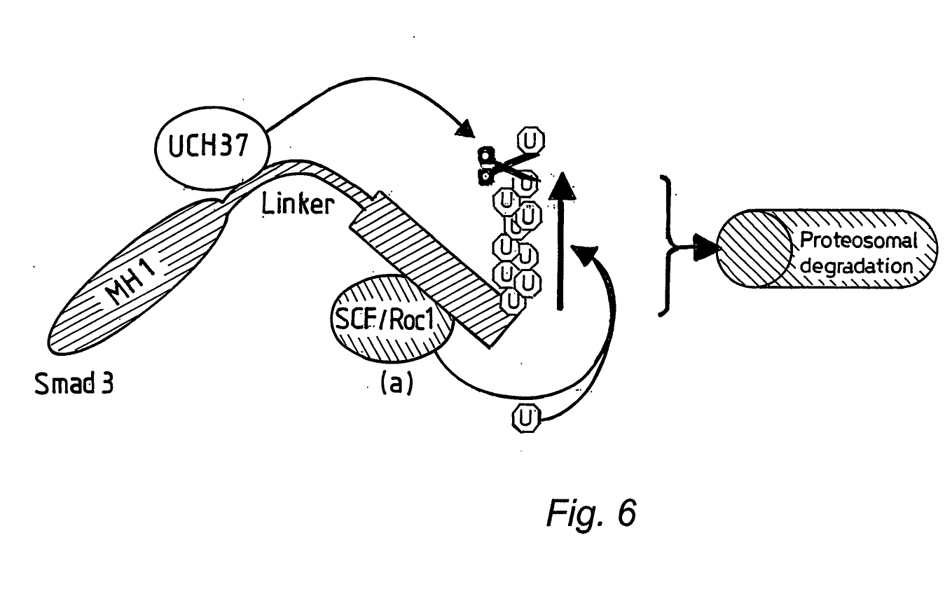

FIG. 6 is a schematic representation of the interaction of UCH37 with Smad3 and how this interaction protects the Smad protein from Ubiquitin-mediated proteasomal degradation; and

FIG. 7 shows the amino acid sequence of human Smad3 (Seq. ID No. 1);

Referring to FIG. 1, in pathway (a), activated TGF-βRI associates with receptor-regulated Smads 2 or 3 (“R-Smad”). Subsequent R-Smad phosphorylation at C-terminal serines leads to hetero-oligomerisation with the common-mediator (“Co-Smad”), Smad 4. The hetero-oligomeric complex is then translocated to the nucleus, where it binds directly, or in complex with other components, to DNA and affects transcription of specific genes. In pathway (b), activated BMP-RI signals in a similar way to TGF-βRI. However it associates with, and causes phosphorylation of, R-Smads 1 or 5 rather than 2 or 3.

Referring to FIG. 2, in step (a), cytoplasmic R-Smad ubiquitination and proteasomal degradation is mediated by Smurfs. In step (b), nuclear activated R-Smads are degraded after Smurf-mediated ubiquitination. In step (c) nuclear R-Smads are ubiquitinated by the action of SCF/Roc 1 E3 ligase complex, exported to the cytoplasm and undergo proteasomal degradation.

Referring to FIG. 6, in step (a) nuclear R-Smads (e.g. Smad3) are ubiquitinated by the action of the MH2 bound SCF/Roc1 E3 ligase complex, exported to the cytoplasm and undergo proteasomal degradation. In step (b), UCH37 which binds to Smad-3 in the region aa 144-240, facilitates the removal of ubiquitin and may prevent targeted proteasomal degradation of the Smad protein.

Smads are segregated into three functional groups. R-smads (Smads-1, 2, 3, and 5), as mentioned above, are directly phosphorylated and activated by the activated TGFβ receptor complex. Co-Smad (Smad-4) associates with activated R-Smads and plays a role in targeting the activated Smad complex to the nucleus. Inhibitory Smads, or “I-Smads” (Smads-6 and 7), are able to down-regulate the TGFβ response mainly by recruiting specific E3 ubiquitin ligases known as Smurfs (Smad ubiquitin regulatory factors). Interestingly, although R-Smads were originally found to bind to Smurfs (Zhu et al, 1999 Nature, 400, 687-693), further studies showed subsequently that interactions between I-Smads and Smurfs could potentially have more physiological relevance. Following prolonged exposure of cells to TGFβ, the I-Smad7/Smurf complex forms, exits the nucleus, binds to the activated TGFβ receptor complex, and the associated E3 ligase (or Smurf) then ubiquitinates the receptors leading to their rapid proteasomal degradation (Kavsak et al, 2002 Molecular Cell 6, 1365-1375). Interactions between I-Smads and a de-ubiquitinating enzyme such as UCH37 could therefore also affect this pathway of down-regulation.

### EXAMPLES

The inventors used a yeast two-hybrid approach to identify proteins that interact with Smad3 and potentially regulate the TGFβ signalling pathway. A mouse brain cDNA library was screened and the positive clones were identified by sequencing and subsequent BLAST DNA database searches. Using this approach, the inventors found a Smad-interacting protein which was identified as a ubiquitin C-terminal hydrolase known as UCH-L5 (Genbank No. NM 019562 or AF148447) in mouse or UCH37 in humans (Genbank No. AF147717).

The yeast two-hybrid materials are commercially available from Clontech Laboratories Inc. (1020 East Meadow Circle, Palo Alto, Calif. 94303, USA). The technique is described in detail in the “MATCHMAKER GAL4 Two-Hybrid System 3 and Libraries User Manual” (PT3247-1 [PR94575]) published by Clontech, June 1999 (see also MATCHMAKER Two-Hybrid System 3, January 1999 CLONTECHniques XIV(1): 12-14).

In their screen, the inventors used sequences comprising residues 1-240 of Smad3 (Smad3₁₋₂₄₀) as bait in the Clontech yeast two-hybrid system. Interacting proteins were then investigated by co-expression and co-immunoprecipitation experiements using epitope-tagged proteins.

In these experiments, a FLAG®-tagged full length UCH-L5 or a truncation lacking a C-terminal extension downstream of the N-terminal enzymatic domain (UCH-L5ΔC lacking residues Trp₁₉₆-Lys₃₂₉) was co-expressed with Haemagglutinin (HA)-tagged Smad proteins. Expression of these proteins was confirmed by western blot and interactions were identified by anti-HA western blot probing of anti-FLAG immunoprecipitates. The experimental techniques used are routine for those skilled in the art and are described, for example, by Sambrook et al. (Molecular Cloning. A Laborarory Manual. 2^{nd} edition. Coldspring Harbor Press, Coldspring Harbor. USA) and by Wicks et al., (2000 Mol. Cell. Biol. 20, 8103-8111).

By way of explanation, the FLAG® tag (FLAG is a registered trade mark of Sigma-Aldrich Biotechnology LP) is a short peptide tag (amino acid sequence DYKDDDDK, Seq. ID No. 2) which is incorporated into proteins expressed using the commercially available pFLAG® expression construct (pFLAG is a registered trade mark of Sigma-Aldrich Biotechnology LP). Monoclonal antibodies are available which are specific for the FLAG® peptide and so can be used to detect FLAG®-labelled proteins. The FLAG® system is further detailed and described in EP 0150126 and EP 0335899.

C-terminally FLAG®-tagged UCH-L5 or UCH-L5ΔC were co-expressed in human embryonic kidney (HEK)—293 cells with N-terminally HA-tagged Smad proteins. The results of the immunoprecipitation experiments are presented in FIGS. 3(i)-(iii).

Referring to FIG. 3, HEK-293 cells were transfected with 20 μg of DNA construct directing the expression of UCH-L5 FLAG or UCH-L5ΔC FLAG and 20 μg of DNA construct expressing one of: HA-Smad 3₁₋₃₈₅; HA-Smad3₁₋₂₄₀; or HA-Smad3₁₋₄₄. Lysates of the cells were prepared by a standard detergent lysis method, using 1% Triton X-100 and then immunoprecipitated with an anti-FLAG monoclonal. The precipitated proteins were then subjected to SDS-PAGE. The results are shown in FIG. 3(iii).

For comparison, a blot of the same samples was then probed with a haemagglutinin-specific 1^{st} antibody. Clear bands were detected corresponding to Smad 3₁₋₃₈₅ or Smad3₁₋₂₄₀; these proteins had therefore been co-immunoprecipitated with UCH-L5 or UCH-L5ΔC, demonstrating that the UCH-L5 or L5ΔC proteins must have been complexed with the Smad3₁₋₃₈₅ or Smad3₁₋₂₄₀ proteins. In contrast, no band could be detected at the position corresponding to Smad3₁₋₁₄₄, indicating that this protein was not bound by UCH-L5 or L5ΔC. From this, the inventors deduced that the binding site for UCH-L5 on Smad3 must encompass at least a portion of the Smad3 protein present between residues 144 and 240.

FIG. 3(ii) shows the results of the control experiment in which whole cell lysates of HEK-293 cells were run on a gel, blotted, and probed with an anti-haemagglutinin 1^{st} antibody. These confirmed that the small Smad3₁₋₁₄₄ truncated protein was being expressed by the cells and therefore the failure to detect the protein in the immunoprecipitated material must have been due to its lack of binding to the UCH-L5 FLAG or UCH-L5ΔC FLAG proteins. The results in FIG. 3 also demonstrate that there is no significant difference between the results for UCH-L5 or for the C-terminally truncated version UCH-L5ΔC.

### Example 2

In the light of the foregoing results the inventors decided to investigate further the interaction between Smad3 and UCH-L5. In particular the inventors wished to discover if over-expression of UCH-L5 could affect levels of Smad-dependent gene expression. To this end, they utilised a plasmid, SBE-luc (Labbé et al, 1998 Molec. Cell 2, 109-120) which expresses the luciferase reporter gene in a Smad-dependent manner, the luciferase-coding sequence being operably linked to a Smad binding element (“SBE”).

HEK-293 cells were transfected with

5 μg SBE-luc alone; or

5 μg SBE-luc+10 μg UCH-L5 FLAG construct; or

5 μg SBE-luc+5 μg TGF-β receptor construct; or

5 μg SBE-luc+10 μg UCH-L5 FLAG construct and 5 μg TGF-β receptor construct.

The TGF-β receptor construct directed the expression of the constitutively active type I receptor [TGF-βRI_{T204D}]).

The resulting level of luminescence, due to luciferase-activity, was assayed the luciferase reporter kit (Roche). The results are shown in FIG. 5, which is a bar chart showing change in luminescence (arbitary units) for cells in groups (a)-(d).

The cells transfected with SBE-luc alone did not generate any luminescence. Co-expression of SBE-luc and UCH-L5 had no significant effect. Co-expression of SBE-luc and TGFβ receptor caused a modest increase in luminescence. Co-expression of SBE-luc simultaneously with both UCH-L5 and TGFβ receptor caused a significant (about 6 fold) further increase in luminescence.

In conclusion the inventors have identified a novel interaction between the Smad3 transcription factor and a ubiquitin C-terminal hydrolase and believe that this interaction could lead to stabilisation of the Smad3 protein and potentiation of TGFβ signalling by reversal of ubiquitin-mediated proteasomal degradation via ubiquitin ligase containing complexes such as SCF/Roc1. Targeting of a specific drug or peptide mimetic to the interaction domain between Smad3 (within residues 144-240) and UCH37 in humans could be useful to treat diseases or conditions, especially those in which there is over-activation of the TGFβ signalling pathway (FIG. 4). Examples of detrimental gain of TGFβ signalling can be found in fibrotic disease, wound healing/scarring, and eye diseases such as cataract. Increases in TGFβ signalling are also thought to play a role in the late stages of cancer in which there is formation of new blood vessels (angiogenesis) that supports tumour growth, and metastatic migration of tumour cells.

### Example 3

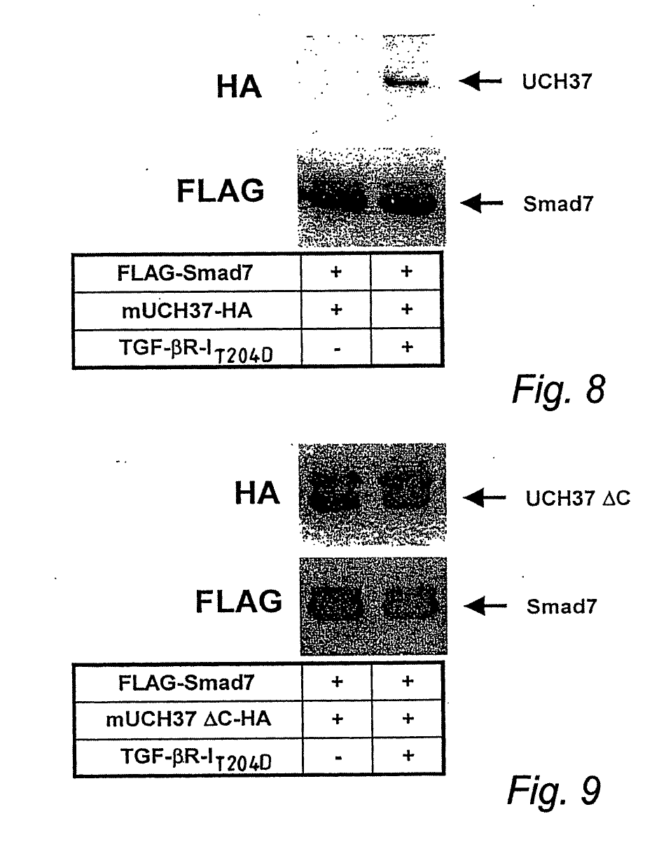

The inventors conducted further experiments to investigate if UCHs could associate with Smads other than Smad3. In particular, the inventors have found that UCH37 binds to the inhibitory Smad, Smad7, and that this interaction is TGFβ-dependent (FIG. 8). In addition, the inventors found that removal of the c-terminal tail in UCH37ΔCT (a construct essentially equivalent to UCH-L5ΔC, described in the preceding examples) leads to an enhanced interaction with Smad7 independently of TGFβ signalling (FIG. 9).

FIGS. 8 and 9 show the results of experiments in which HEK-293 cells were transfected with 15 μg of DNA construct directing the expression of Smad7-FLAG and UCH37-HA (FIG. 8) or UCH37ΔCT-HA (FIG. 9) in the presence (+) or absence (−) of 15 μg of a DNA construct directing the expression of activated [TGFβRI_{T204D}]. Lysates of cells were prepared by a standard detergent lysis method, using Triton X-100 (as described in the preceding examples) and then immunoprecipitated with an anti-FLAG antibody. The precipitated proteins were then subject to SDS-PAGE and probed with an anti-HA antibody or anti-FLAG antibody, again as described previously.

FIG. 10 is a bar chart showing results obtained (using protocols as described in relation to the data presented in FIG. 5) when HepG2 cells were transfected with;

0.5 μg SBE-luc alone; or

0.5 μg SBE-luc+16 hours TGFβ (5 ng/ml); or

0.5 μg SBE-luc+500 ng UCH37 HA construct; or

0.5 μg SBE-luc+500 ng UCH37 HA construct+16 hours TGFβ (5 ng/ml); or

0.5 μg SBE-luc+500 ng catalytically inactive UCH37_{C88A} HA construct; or

0.5 μg SBE-luc+500 ng catalytically inactive UCH37_{C88A} HA construct+16 hours TGFβ (5 ng/ml)

The resulting level of luminescence, due to luciferase, was assayed using the luciferase reporter kit (Roche). The bar chart shows change in luminescence in arbitrary units. Co-expression of UCH37 caused a significant decrease in TGFβ-dependent activation of SBE-luc. This response was reversed by replacing UCH37 with a catalytically-inactive mutant, UCH37_{C88A}. These data demonstrate that, in HepG2 cells, UCH37 can down-regulate TGFβ signalling.

It is feasible that an I-Smad such as Smad7 bound to UCH37 could stabilize Smad7 by de-ubiquitination and therefore encourages the Smad7-dependent downregulation of TGFβ receptor signalling. Alternatively, Smad7 bound to UCH37 could lead to de-ubiquitination of the associated receptor complex, and thereby promote TGFβ signalling. The inventors' experimental data suggest that both mechanisms could occur in a very cell-type specific manner, and that blockade of the UCH37/Smad7 interaction could, in some instances, down-regulate TGFβ responses (HEK-293 fibroblasts; see FIG. 5), and in a different cellular context it could up-regulate TGFβ responses (HepG2 cells; FIG. 10). In summary, blockade of interactions between Smad3 and UCH37 and/or Smad7 and UCH37 could provide therapeutic benefit in diseases in which there is either down- or up-regulation of TGFβ signalling.

1. A pharmaceutical composition for altering cellular responses to TGFβs and/or BMPs; the composition comprising a molecule which prevents, inhibits or reduces the association of a Smad protein with a UCH, or a nucleic acid construct directing the expression of such a molecule, in admixture with a physiologically acceptable carrier, excipient or diluent.

2. A composition according to claim 1, wherein the composition prevents, inhibits or reduces the association of a Smad3 protein with a UCH.

3. A composition according to claim 1, wherein the composition prevents, inhibits or reduces the association of a Smad protein with UCH37.

4. A composition according to claim 1, wherein the composition comprises, as an active agent, a molecule which comprises a structural analogue of the UCH-binding site of a Smad protein.

5. A composition according to claim 1, wherein the composition comprises, as an active agent, a molecule which comprises a structural analogue of the Smad-binding site on a UCH protein.

6. A composition according to claim 1, wherein the active agent comprises a peptide of at least 8 amino acid residues which exhibits at least 60% identity, preferably at least 70%, more preferably at least 80%, and most preferably at least 90% identity, with a contiguous portion of a Smad polypeptide; or a nucleic acid construct directing the expression of such a peptide.

7. A composition according to claim 6, wherein the peptide comprises at least 10 residues.

8. A composition according to claim 6, wherein the peptide comprises at least 12 amino acid residues.

9. A composition according to claim 6, wherein the peptide comprises at least 15 amino acid residues.

10. A composition according to claim 6, wherein the peptide comprises fewer than 80 amino acid residues.

11. A composition according to claim 10, wherein the peptide comprises fewer than 60 amino acid residues.

12. A composition according to claim 10, wherein the peptide comprises fewer than 40 amino acid residues.

13. A composition according to claim 6, wherein the peptide exhibits at least 60% sequence identity with a contiguous portion of Smad3.

14. A composition according to claim 6, wherein the peptide exhibits at least 60% identity with a contiguous portion of Smad3 present within amino acid residues 114-240 thereof.

15. Use of a substance which prevents, inhibits or reduces the association of a Smad protein with a UCH, in the preparation of a medicament to cellular responses to TGFβs and/or BMPs.

16. Use of a substance, in accordance with claim 15, in the preparation of a medicament in accordance with claim 1.

17. A method of altering cellular responses to TGFβs and/or BMPs, the method comprising the step of introducing into a cell a molecule which prevents, inhibits or reduces the association of a Smad protein with a UCH.

18. A method according to claim 17, which comprises the step of administering a composition in accordance with claim 1.

19. A method of screening a test substance for the ability to prevent, inhibit or reduce the association of a Smad protein with a UCH, the method comprising the step of contacting the test substance with a Smad protein and/or a UCH and determining, qualitatively or quantitatively, the amount of association of the Smad protein with the UCH when these are contacted.

20. A method according to claim 19, wherein at least one of the test substance, Smad protein and UCH is labelled with a readily detectable label.
